(19) United States
(12) Patent Application Publication
(10) Pub. No.: US 2002/0154269 A1
(43) Pub. Date: Oct. 24, 2002

(54) Stereoscopic measurement of cornea and illumination patterns

(76) Inventors: David Liu, Winter Springs, FL (US); Edwin J. Sarver, Merritt Island, FL (US); Dale Troendle, Lake Mary, FL (US)

Correspondence Address: MANELLI DENISON & SELTER PLLC, 2000 M Street, N.W., 7th Floor, Washington, DC, 20036-3307, US

(21) Appl. No.: 10/122,239

(22) Filed: Apr. 16, 2002

Related U.S. Application Data
(60) Provisional application No. 60/283,625, filed on Apr. 16, 2001.
(60) Provisional application No. 60/283,627, filed on Apr. 16, 2001.

(51) Int. Cl. A61B 3/14

(52) U.S. Cl. 351/206

(57) ABSTRACT

A stereoscopic eye measurement system and method for measurement of corneal characteristics, anterior chamber depth and lens characteristics in a single acquisition. The system and method use a stereoscopic camera configuration to capture the images of IR pupil, of the intersection of a structured illumination pattern with the cornea and lens, and of the Placido reflection off the cornea. The projection pattern may be a cross pattern, a dot array, a dot+cross pattern, or a starburst pattern. The system uses a large pupil in order to obtain images of the lens. The system uses different focal points to achieve the best images of corneal topography, corneal layering and lens surfaces and a combination of corneal topography, corneal layering, pupil and the lens.

This application claims priority from U.S. Provisional Appl. No. 60/283,625, entitled “Stereoscopic Measurement of Corneal Thickness, Anterior Chamber Depth, Thickness Of The Intra-Ocular Lens And/Or The Curvature Of The Lens And The Opacity of the Lens,” and U.S. Provisional Appl. No. 60/283,627, entitled “Illuminating Pattern for the Cornea, Anterior Chamber and Intra-Ocular Lens Measurement Using Stereo Imaging of One of a Few Rapid Acquisitions,” both filed on Apr. 16, 2001, as well as from U.S. Appl. Ser. No. 09/860,558, entitled “Combination Advanced Topography/Wave Front Aberration Measurement”, filed May 21, 2001, which claims priority from U.S. Pat. No. 6,234,631, entitled “Combination Advanced Corneal Topography/Wave Front Aberration Measurement” to Sarver et al., filed Mar. 9, 2000, and from U.S. Appl. Ser. No. 09/978,657, entitled “Stereo View Reflection Corneal Topography”, filed Oct. 18, 2001, claiming priority from U.S. Provisional Appl. No. 60/240,983, entitled “Stereo View Reflection Corneal Topography”, filed Oct. 18, 2000, the entirety of all of which are expressly incorporated herein by reference.

### BACKGROUND OF THE INVENTION

1. Field Of The Invention

This invention relates generally to non-destructive measurement of characteristics of the eye's cornea, anterior chamber depth and intraocular lens. More specifically, it relates (1) to stereoscopic measurement of corneal thickness curvature and topography, anterior chamber depth, and intraocular lens thickness, curvature and opacity; and (2) to illumination patterns used in these measurements.

2. Background

Quality of vision depends on a number of elements of the eye. One such element is the cornea, which is the front surface of the eye and provides about two-thirds of the eye's refractive power. Another is the depth of the anterior chamber, which is the distance between the intraocular lens' front surface and the cornea. A third exemplary element is the intraocular lens, which further focuses light coming through the pupil onto the retina.

Accurate measurements of the cornea, anterior chamber depth and intraocular lens and their characteristics are of great concern in the field of ophthalmology and optometry. The accuracy of these measurements directly affects the ability to detect early corneal and lens disease, to compute the correct power for a phakic or aphakic intraocular lens, and/or to perform surgery successfully to correct corneal and lens conditions.

Existing methods of making measurements of the cornea, anterior chamber and lens use a single slit or scanning slit method. FIG. 18 shows a conventional single slit or scanning slit method.

In particular, as shown in FIG. 18, an illumination source (not shown) illuminates a target 1810 to create a placido (a structured illumination pattern), which is then projected onto the cornea 1812 of a patient's eye 1814. The cornea in part reflects the rays. A front view camera with lens 1806 captures the rays and focuses them onto a CCD 1804. A computer 1802 processes the image to generate measurements of the cornea's anterior topography.

A single slit or scanning slit projects a illuminated slit pattern on to the cornea. The center camera captures the slit image(s). And a computer program analyzes these images to get the measurement of the corneal thickness, etc.

Existing single slit or scanning slit methods result in some inaccuracies in measurements. In the case of scanning slit, for example, multiple images are captured during a time interval to capture the whole eye. Patient and eye motions, however, tend to cause inaccuracy and alignment error in the captured data. Moreover, the measurement data acquisition is time consuming and because of the length of time can cause patient discomfort due to bright light alignment, focusing and acquisition. Furthermore, the bright light alignment and focusing during the examination process causes the patient's pupil to contract, preventing the instrument to obtain significant imaging of the lens.

There is a need for an improved technique and apparatus for measurement of corneal thickness and curvature, anterior chamber depth and/or lens characteristics that allows quick and accurate data acquisition.

### SUMMARY OF THE INVENTION

An improved reflective illuminated target system and method that uses stereo optics to generate data about corneal thickness, curvature and topography, pupil contour, size and location, anterior chamber depth, and lens thickness, curvature and opacity. The patient's eye alignment and focusing are performed under infrared illumination, which allows a large pupil to facilitate the imaging of the lens. Stereo optics allows the capture of all data using only one or a few exposures, thereby increasing accuracy and decreasing patient discomfort. Patterns that are suitable for stereo imaging are, e.g., a cross pattern, a cross+dot array, a dot array, and a starburst pattern (cross and X pattern).

### BRIEF DESCRIPTION OF THE DRAWINGS

Features and advantages of the present invention will become apparent to those skilled in the art from the following description with references to the drawings, in which:

FIGS. 1A and 1B show top and side views of an exemplary embodiment of a stereoscopic placido system, in accordance with the principles of the present invention.

FIG. 2 shows an exemplary cross pattern that is suitable for stereo imaging, in accordance with the principles of the present invention.

FIG. 3 shows an exemplary cross+dot array that is suitable for stereo imaging, in accordance with the principles of the present invention.

FIG. 4 shows an exemplary dot array that is suitable for stereo imaging, in accordance with the principles of the present invention.

FIG. 5 shows an exemplary starburst pattern (cross and X pattern) that is suitable for stereo imaging, in accordance with the principles of the present invention.

FIG. 6 shows one of two side views obtained from the stereoscopic placido system shown in FIGS. 1A and 1B from a camera at 30 degrees from center, using a 250 micron cross.

FIG. 7 shows a front view image obtained from the stereoscopic placido system shown in FIGS. 1A and 1B using a 150 micron cross.

FIG. 8 shows one of two side views obtained from the stereoscopic placido system shown in FIGS. 1A and 1B from a camera at 30 degrees from center using a 150 micron cross.

FIGS. 9A and 9B show an exemplary cross pattern image method.

FIG. 10 shows an exemplary configuration for obtaining a front view pupil image and a side view pupil image at different angles.

FIGS. 11A-11C show exemplary pupil images viewed from the front, 30 degree angle and 45 degree angle respectively, in accordance with the principles of the present invention.

FIGS. 12A to 12E show an exemplary use of stereo infrared pupil contours to locate the focal point using the measurement system of FIGS. 1A and 1B.

FIG. 13 shows an exemplary use of the stereoscopic system of FIGS. 1A and 1B to measure corneal layers.

FIG. 14 shows an exemplary use of the stereoscopic system of FIGS. 1A and 1B to capture measurement of corneal topography focusing.

FIG. 15 shows an exemplary use of the stereoscopic system of FIGS. 1A and 1B to capture measurement of the intraocular lens.

FIG. 16 shows an exemplary use of the stereoscopic system of FIGS. 1A and 1B to improve cross-pattern projection from a skew angle.

FIG. 17 shows an exemplary use of the stereoscopic system of FIGS. 1A and 1B to capture the whole eye.

FIG. 18 is a representation of a conventional monocular placido system.

### DETAILED DESCRIPTION OF ILLUSTRATIVE EMBODIMENTS

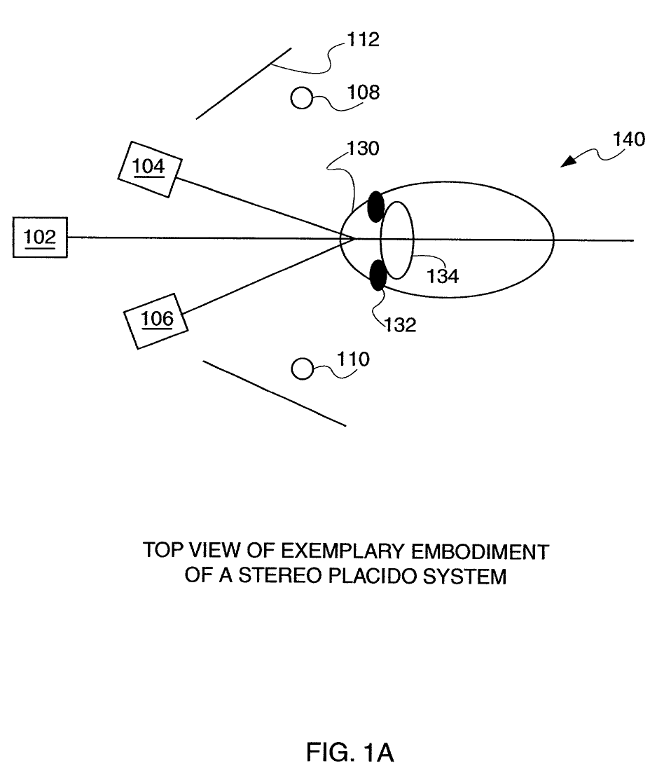

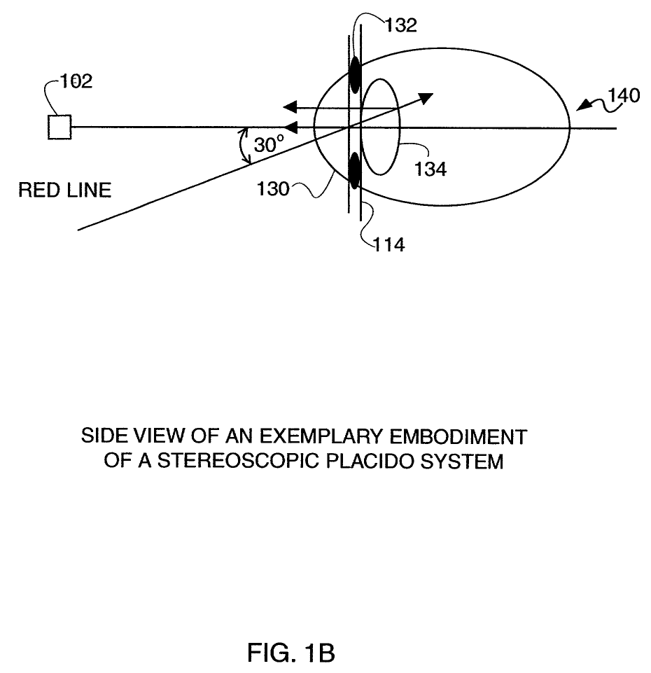

FIGS. 1A and 1B show an exemplary embodiment of a stereoscopic placido target system, in accordance with the principles of the present invention.

In particular, FIG. 1A shows a top view of the setup. Although only a center and one side camera are required at a minimum, three cameras are shown and preferred: a center camera 102 and two side cameras 104 and 106 that are at skewed angles to the eye 140. The cornea 130, iris/pupil 132 and lens 134 of the eye 140 are schematically shown.

It is preferred that a large pupil size be obtained, because a large pupil size is a key to the present invention. For instance, the pupil size should be a minimum of 4.5 mm for measurement. As the pupil size becomes larger, the lens becomes more exposed, which improves the accuracy of the measurement. Infrared (“IR”) illumination allows a large pupil, although of course a pupil may be dilated to obtain the proper size.

In accordance with the principles of the present invention, two IR illumination sources 108 and 110 are used at angles to the eye 140 to illuminate the measured surface. This IR illumination is to facilitate the alignment and focusing process as well as providing illumination for pupil size measurement. A pulsed light source illuminates a Placido 112, which has a structured pattern such as concentric rings or polar grid pattern. The pulse-illuminated Placido pattern is reflected off the cornea and the images captured by all cameras for anterior corneal topography measurement. As discussed below, a light source with a structured pattern (not shown) is projected through an opening on a placido 112 onto the cornea 130 and lens 134. All cameras capture the images caused by intersection of the projected light pattern with the corneal layer and the lens.

As shown in FIG. 1B, the system is focused and aligned to enter through the pupil plane. In particular, a center point of the front camera 102 focal plane 114 is the focal point of all three cameras 102, 104 and 106. The pupil 132 is the window for imaging the lens 134.

A pulse of focused cross light pattern (an exemplary structured pattern) 112 is projected onto the cornea 130 and passed through the cornea 130 to hit the lens 134. The stereoscopic camera configuration with cameras 102, 104 and 106 captures the reflection of the image of the intersection of the light pattern with the cornea 130 and the lens 134. Preferably, the cross projection optics has a large depth of focus. It is also preferred that the image optics are designed with a large depth of field as well.

### Illumination Patterns

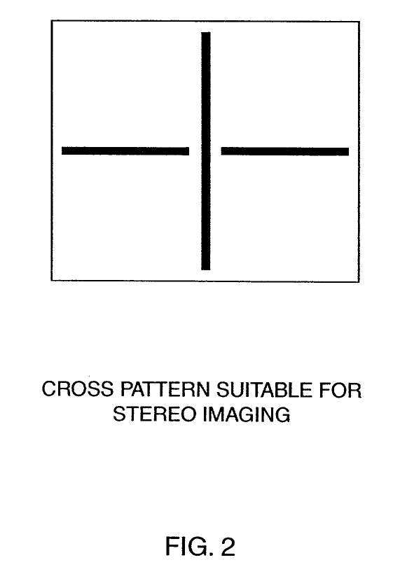

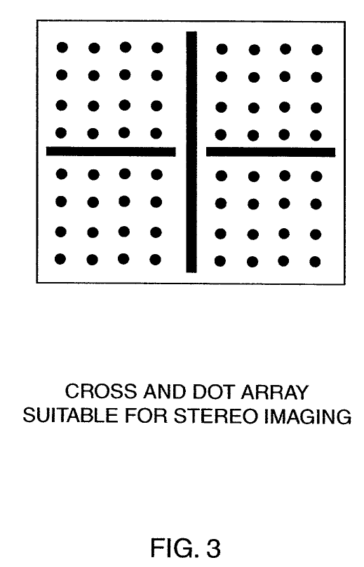

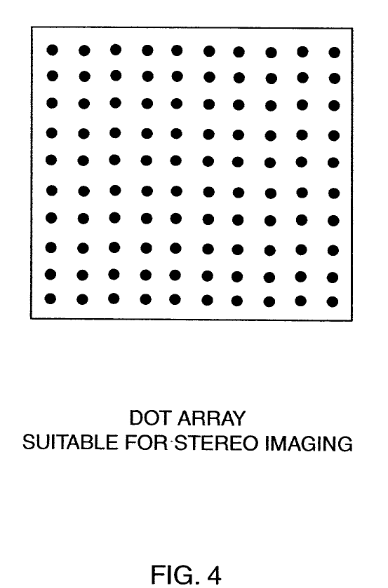

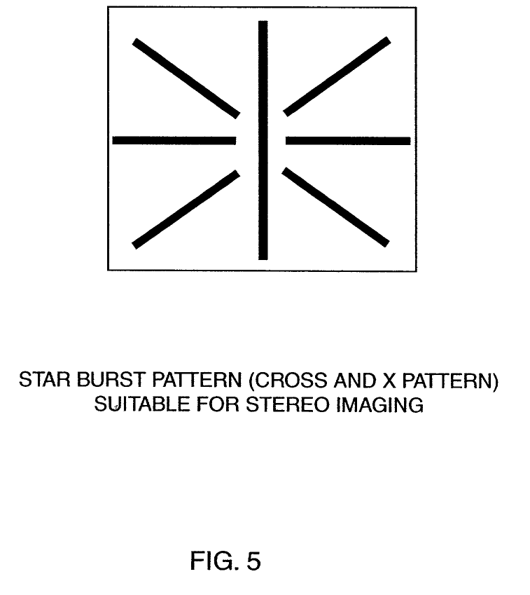

Certain illumination patterns are suitable as the structured projection illuminated pattern shown in FIG. 1A for stereo imaging through one or a few exposures to make measurements in accordance with the principles of the present invention. For instance, FIGS. 2 through 5 shows exemplary representations of these patterns. In particular, FIG. 2 shows an exemplary cross pattern, FIG. 3 an exemplary cross+dot array, FIG. 4 an exemplary dot array, and FIG. 5 an exemplary starburst (cross and X) pattern. Each of these patterns may be used as a structured projection illumination pattern in addition to a Placido in the stereoscopic placido system shown in FIGS. 1A and 1B.

The Placido in FIG. 1A is electronically pulse illuminated to allow the measurement of the anterior corneal topography. The structured projection illumination pattern is pulsed, normally through a electro-mechanical shutter, to allow for the measurement of the cornea layers, anterior segment and lens. The pulse mode illumination for both Placido and projection allows the pupil to remain large under a dark ambient condition to enable the imaging of the lens and measurement of the lens thickness and curvature. The two pulses from Placido and projection are rapid in time sequence (typically a fraction of a second apart, not enough for pupil to contract). In practice, the pulse from the projection pattern cab be imaged first and the Placido second, ensuring maximum possible pupil opening without dilation for optima a corneal layer and lens imaging and measurement.

The cross pattern shown in FIG. 2 allows continued measurement of thickness and curvature along two meridians of both a cornea 130 and a lens 134, as discussed below. It also allows for the measurement of anterior chamber depth and the angle between the cornea 130 and iris 132 at their juncture, as also discussed below.

The dot array shown in FIG. 4 has the advantage of using discrete points intersecting the cornea 130, iris 132 and lens 134, which provide local thickness measurements with higher spatial resolutions. Stereo interrogation of these intersections yields 3-D local positions. Reconstruction algorithms, similar to the one set forth below for the cross pattern, provide a complete estimate of the corneal anterior and posterior curvature and thickness profile, and lens thickness and curvature measurements. The algorithms also allow for the estimate of anterior chamber depth and the angle between the cornea 130 and the iris 132 at their juncture.

The dot+cross array shown in FIG. 3 combines the advantages of both the cross pattern and dot array shown in FIGS. 2 and 4, offering more direct measurement data points than the cross pattern and dot array separately. A suitable reconstruction algorithm provides a complete measurement of the desired eye characteristics.

The starburst pattern shown in FIG. 5 offers improved continuous sampling density, allowing for reconstruction algorithms to provide complete measurement and estimation of the characteristics as is enjoyed by the use of the dot array pattern shown in FIG. 4.

As the diameter of the pattern opening becomes finer, the illuminated layers on the eye become better defined. However, as the pattern opening narrows, the light sources generally must be made more powerful. For the purpose of measurement of the cornea 130, iris 132 and lens 134, the preferred diameter of the pattern opening is between 100 and 300 microns. Of course, the principles of the present invention relate equally to narrower or wider opening measurements.

The pattern targets can be made using any fine line technique, for example photo etching or laser cutting technology. Preferably, the pattern target material should be as thin as possible, typically 100 to 200 microns in thickness and should be less than the pattern opening diameters, to eliminate or reduce diffraction.

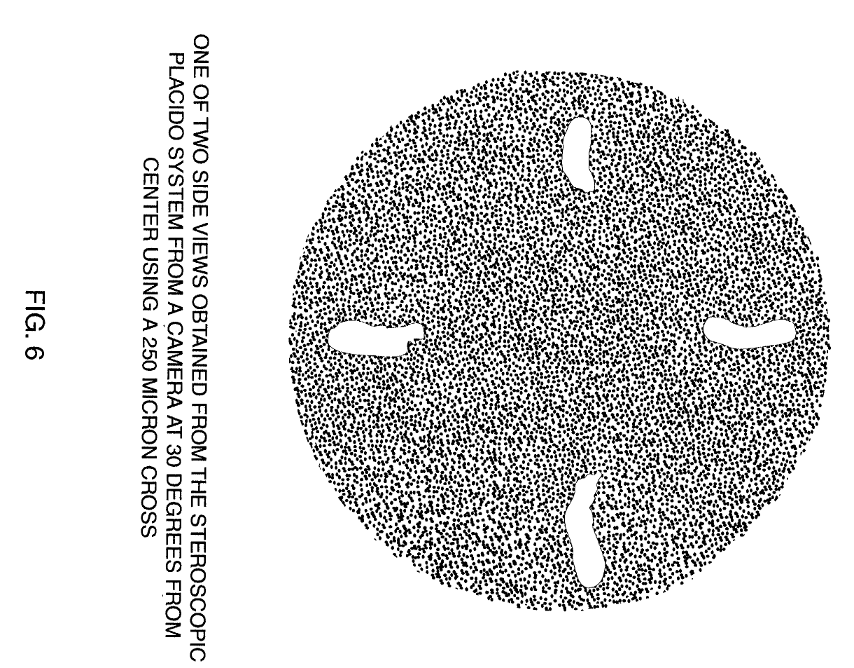

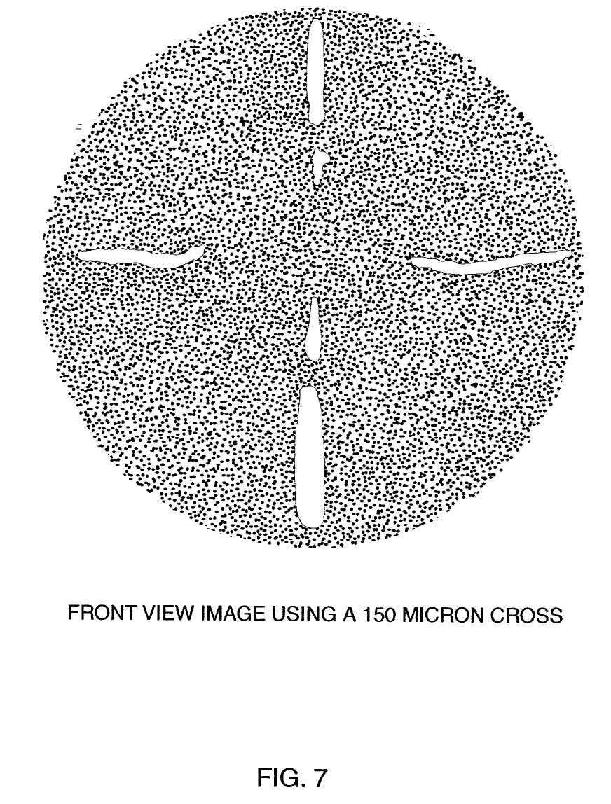

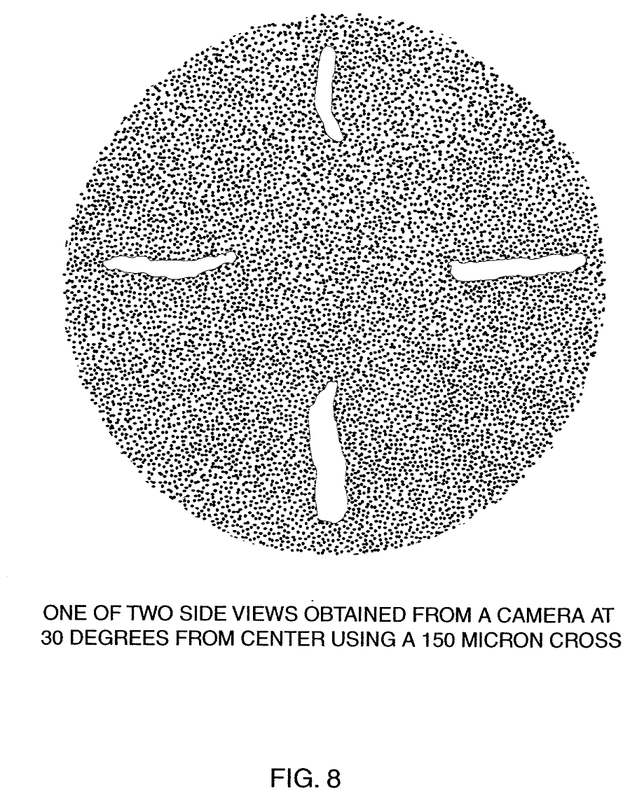

FIGS. 6, 7 and 8 show exemplary images obtained from using cross pattern targets with the stereo imaging system shown in FIGS. 1A and 1B. In particular, FIG. 6 shows one of two side views obtained at 30 degrees from the center using a 250 micron cross pattern target. FIG. 7 shows a front view image using a 150 micron cross target. FIG. 8 shows one of the two side views obtained at 30 degrees from the center using a 150 micron cross pattern target.

### Processing of Stereo Pupil Images

Preferably, a sequence of the stereo images of a target is obtained under variable ambient lighting changing from dark (scotopic) to very bright (photopic). For each lighting condition, preferably three stereo images are obtained. In the given embodiment, from the center view image, the pupil contour is detected and the centroid of the contour, defined as the pupil center, is calculated. This location is recorded relative to the center of the captured image, i.e., the optical axis of the center view image.

Preferably, each of the two side view pupil images is then processed to find the pupil contour of the generally elliptical shape. The centroid, again defined as the pupil center, for each side view is calculated. The pupil centers from the side view images are recorded relative to the center of the captured digital image, defined as the optical axis of each of the side view imaging systems.

The set of data obtained from processing the stereo pupil images is compared with the calibration data obtained when the device is calibrated by imaging a known planar target. This known target is imaged at best focus and away from best focus, too far and too near, off center along the x direction and off center along the y direction, preferably all by a known amount. The recorded target image is then analyzed and a calibration table is generated.

The basic processing algorithm for pupil contour detection is by a box filter along each meridian, starting from an approximate center of the pupil, which is known by the machine when visually aligned by the operator.

The comparison and interpolation gives the location of the pupil contour plane along the Z direction, as well as pupil size. When the pupil changes in diameter, the center and contour of the pupil changes as well. The change of center is significant as it relates to the eye's performance. The results can be printed and graphed to show the pupil's dynamic changes.

### Cross Pattern Image Processing Algorithms

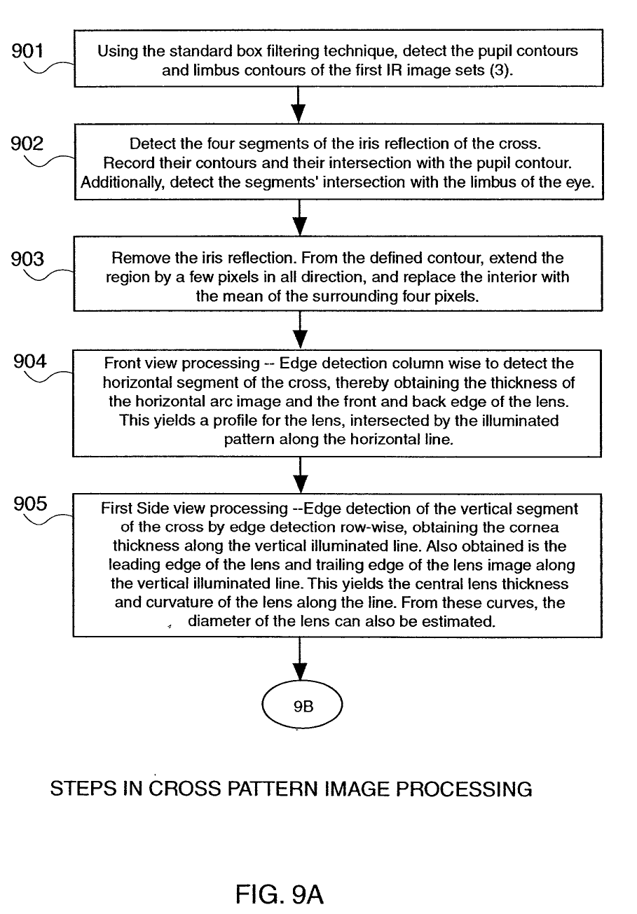

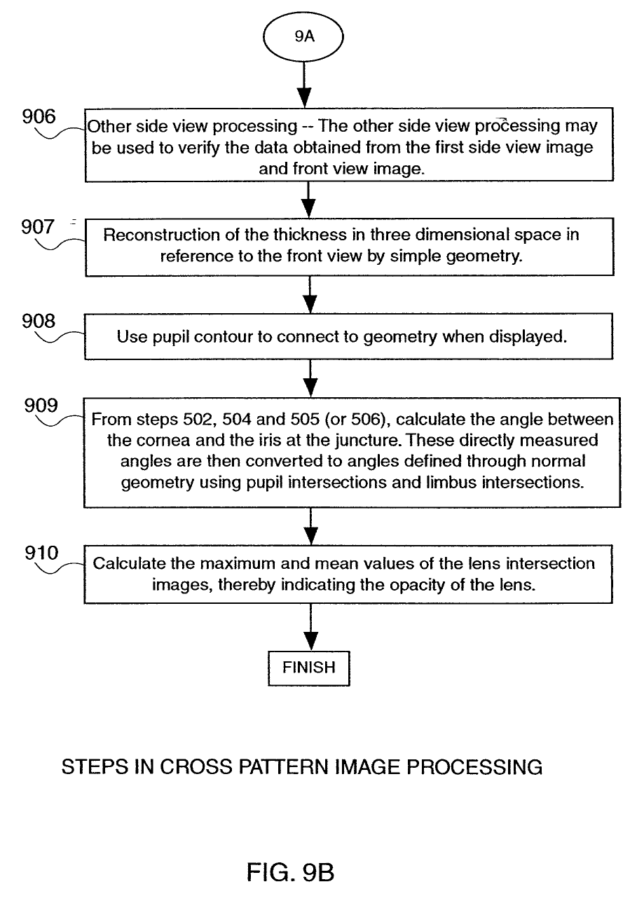

FIGS. 9A and 9B show an exemplary process of processing data from the projection of an exemplary target, e.g., a cross pattern image such as is shown in FIG. 2.

In step 901, the pupil contours and limbus contours of the first IR image sets (3) are detected using the standard box filtering technique.

In step 902, the four segments of the iris reflection of the cross are detected and their contours and their intersection with the pupil contour are recorded. In addition, the segments' intersection with the limbus of the eye is detected.

In step 903, the iris reflection is removed by extending the region from the defined contour by a few pixels in all direction, and replacing the interior with the mean of the surrounding four pixels.

In step 904, the front view is processed. The horizontal segment of the cross is detected by edge detection column wise, thereby obtaining the thickness of the horizontal arc image and the front and back edge of the lens.

This yields a profile for the lens, intersected by the illuminated pattern along the horizontal line.

In step 905, the first side view is processed by edge detection row wise of the vertical segment of the cross. This obtains along the vertical illuminated line the cornea thickness, and leading and trailing edges of the lens image. This in turn yields the central lens thickness and lens curvature along the vertical line. From these curves, the diameter of the lens can also be estimated.

In step 906, the other side view is processed to obtain data to verify the data obtained from the first side view image and front view image.

In step 907, the thickness is reconstructed in three dimensional space in reference to the front view by simple geometry.

In step 908, the pupil contour is used to connect to geometry when displayed.

In step 909, from steps 902, 904 and 905 (or 906), the angle between the cornea and the iris at the juncture is calculated. These directly measured angles are then converted to angles defined through normal geometry, using pupil intersections and limbus intersections.

In step 910, the maximum and mean values of the lens intersection images are calculated, thereby indicating the opacity of the lens.

### Focusing Mechanism Under IR Illumination of the Eye

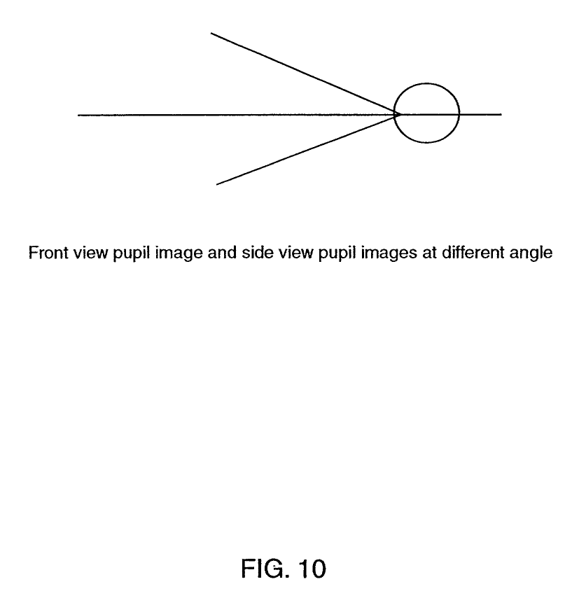

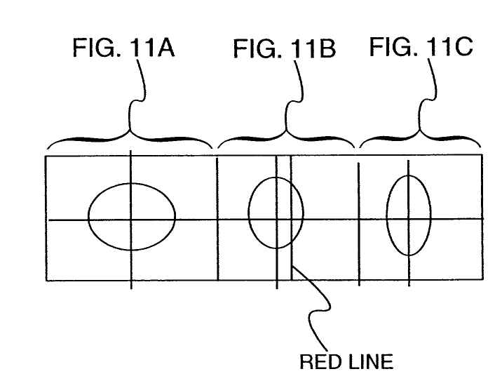

Stereoscopic viewing of the pupil contour facilitates focusing of the cameras 102, 104, and 106 (shown with camera angles of +/−30 degrees). FIG. 10 shows an exemplary pupil image and side view pupil images taken at different angles. FIGS. 11A to 11C show pupil images viewed from the front, from a side angle at 30 degrees, and from a side angle at 45 degrees, respectively.

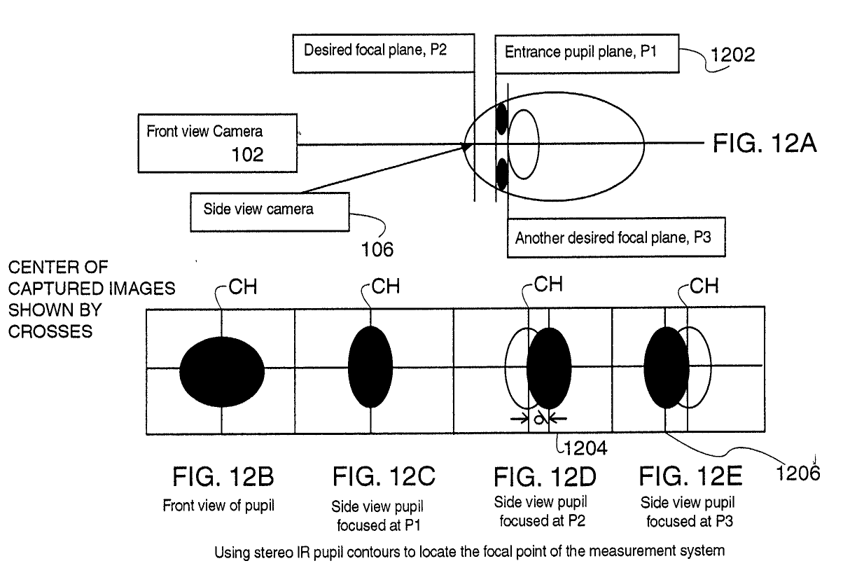

FIGS. 12A to 12E illustrate the ability to focus the system anywhere that is a few millimeters (mm) in front of or behind the entrance pupil plane 1202 by aligning the stereo pupil images to a pre-defined reference target on the computer screen. This approach is good for at least delta =+/−3 mm.

The entrance pupil plane 1202 for a typical eye is 3.05 mm behind the apex of the cornea. In FIGS. 12A to 12E, all the crosshairs CH are the centers of the captured images.

The front view pupil image is always centered about the cross hairs CH regardless of the exact focal plane. Only the pupil image detail changes with focusing. The side view image, when focused at the entrance pupil plane 1202, will be centered on the cross hair. When the side view image is focused in front of or behind the entrance pupil plane 1202, however, the center of the side view pupil image is displaced by an amount d that can be calculated according to a formula, which for an exemplary side view with a camera skewed thirty degrees from the optical axis of the front view camera would be:

Displacement horizontally of side view image=focus_deltatan(30)cos(30)=focus_delta sin(30).

### Focusing For Best Corneal Layer Imaging

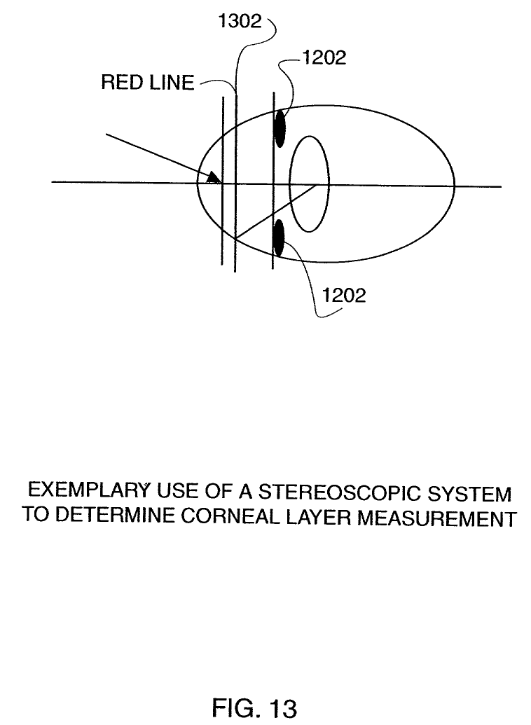

FIG. 13 shows use of the system of FIGS. 1A and 1B in order to obtain optimal corneal layer imaging. In FIG. 13, the focus is not set at the entrance pupil plane 1202 (3.05 mm behind the cornea apex), but rather somewhere between the apex and the entrance pupil plane 1202. The reason is that the central cornea is about 3 mm away from the entrance pupil plane 1202.

Assuming a radius of 8 mm for a typical cornea, a 10 mm diameter corneal chord will give an apical distance of 1.755 mm. To optimally capture the 10 mm cornea, the focal plane is preferably set at the red line 1302 of FIG. 13, the middle of the apical distance, i.e. 1.755/2=0.88 mm from the apex. Therefore, the focal plane from the entrance pupil plane 1202 is 2.17 mm (3.05 mm−0.88 mm) in front of the entrance pupil plane 1202. As an example, with 30 degree stereo viewing, this translates into 1.0825 mm lateral movement of the center of the pupil according to the above equation. This is converted to pixels based on the magnification of the optics and image resolution. The resulting offset may be programmed into the relevant software function to offset the crosshair CH reference marks (green) 1204 and 1206 as shown in FIGS. 12A to 12E.

Thus, for corneal layer measurement only, it is preferable to focus the cross at the corneal plane 1302, i.e. the red line of FIG. 13, from apex to peripheral. In the given example, the red line is 0.88 mm away from the corneal apex.

### CT Capture Focusing

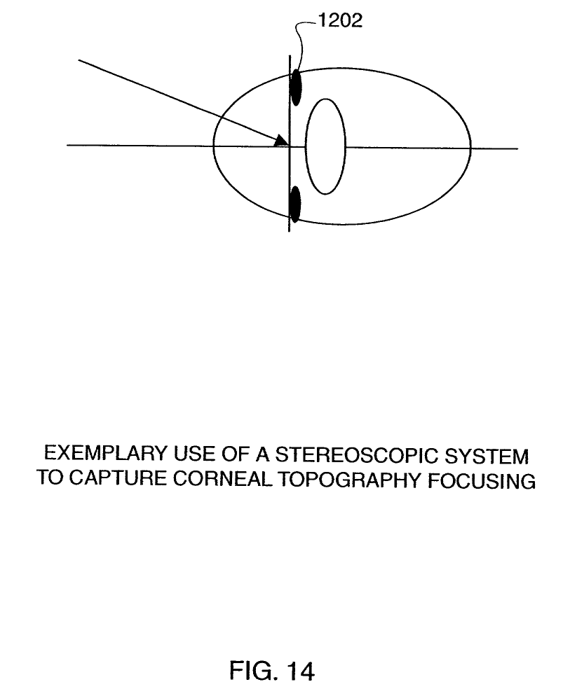

FIG. 14 shows focusing the system of FIG. 1A and 1B to capture corneal topography (CT).

As shown in FIG. 14, the system is preferably focused using the entrance pupil plane 1202, with no offset for the pupil center. At this position, the cross will cut lower on the cornea.

### Focusing To Capture Lens Alone

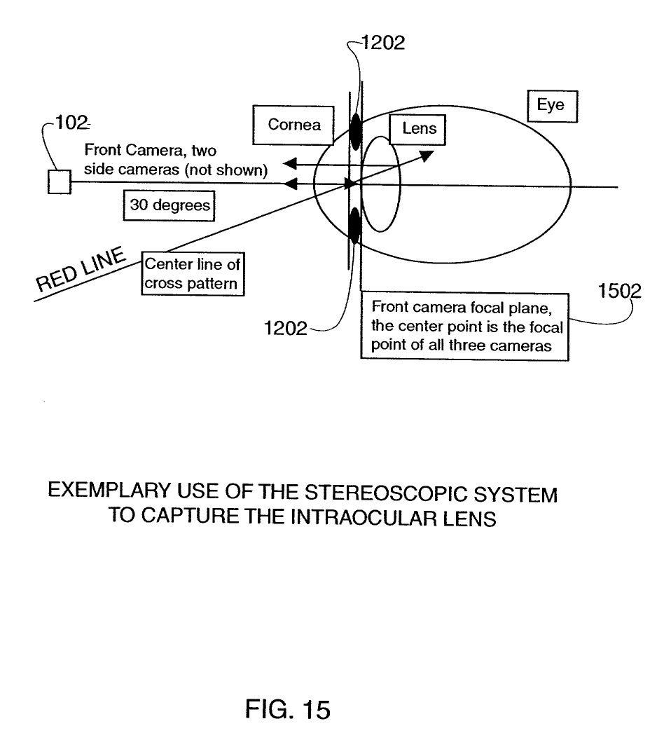

FIG. 15 shows focusing the system of FIG. 1A and 1B to obtain the best quality capture of lens data. For this purpose, the image pattern (i.e., the cross pattern) is aligned and focused at the lens front surface plane 1502. This sets the focal plane at about 3.5 mm behind the apex. The cross should be aligned to intersect the lens front surface at the center horizontally. The back surface of the lens will be imaged when the pupil is large enough.

FIG. 15 depicts the relationship. The cross pattern (only the center light ray is drawn, the red line) is focused at the lens front surface 1502, intersecting close to the center of the lens front surface and intersecting again with the lens back surface at a higher position. All three cameras 102, 104 and 106 capture the two intersections of the light with the lens surface, as well as the scattering (if any) in between. For a clear lens, the gradient change causes certain scattering to be imaged and captured. For a cataract lens, the opacity is captured. Image analysis of captured images provides a good quantitative measure of a cataract.

The ability to capture the back surface of the lens depends on the size of the pupil and also where the stereo image pattern (e.g., cross pattern) hits the lens. Using the center ray of the cross pattern as an illustration, projected from 30 degrees below, and assuming a central lens thickness of 3.6 mm, the minimum pupil size that still allows for the image of the back surface of the lens is 3.6 tan (30)=2.1 mm in radius, which calculates to a 4.2 mm diameter pupil. A minimum pupil size of 4.5 mm diameter pupil is preferred to allow for any misalignment and the image processing need for the intersection. Of course, the larger the pupil size is, the better.

It is also noted that if the angle is reduced from 30 degrees to 15 degrees, the pupil size requirement is smaller, but the accuracy in the stereo measurement of depth (i.e., thickness) suffers as the stereo angle decreases.

Other features of the eye may interfere with applying the image pattern (e.g., cross pattern) on the eye from below. For instance, the lower eyelid and/or eyelash may block the beam if the angle is large enough. Accordingly, it is preferred that the maximum stereo angle should not exceed 45 degrees.

### Global Focusing for Multiple Segment Acquisition

If it is desired to capture the lens surface from the same exposure, it is preferred that the focal point be moved closer to the lens. Furthermore, if it is also desired to capture the corneal topography from the same three cameras at the same time (meaning using the same focusing of the system), the focal point for the corneal topography is best accomplished at about 4 mm behind the cornea apex (for a 8-mm apical radius cornea). Considering all the parameters, it is determined that the best compromise for acquiring corneal layering, lens data and corneal topography is to use a focal point of approximately 3 mm after the apex of the cornea, i.e.,at approximately the entrance pupil plane.

In this case, the image pattern (e.g., cross pattern) is focused at the same plane of the three cameras' focal plane. At a 30 degree angle shooting up, the cross pattern, focused at the same plane as the camera focal plane, will intersect the cornea at approximately 1.5 mm below the center horizontal meridian (center point of the cross). The cross will hit the entrance pupil plane at the center of the pupil plane (at the center of the cross, which is not seen on the iris reflection). The intersection of the cross with the lens' front surface is also at the center of the pupil contour from the front view since the lens is sitting right behind the iris.

### Improvement Image Pattern Projection From a Skew Angle

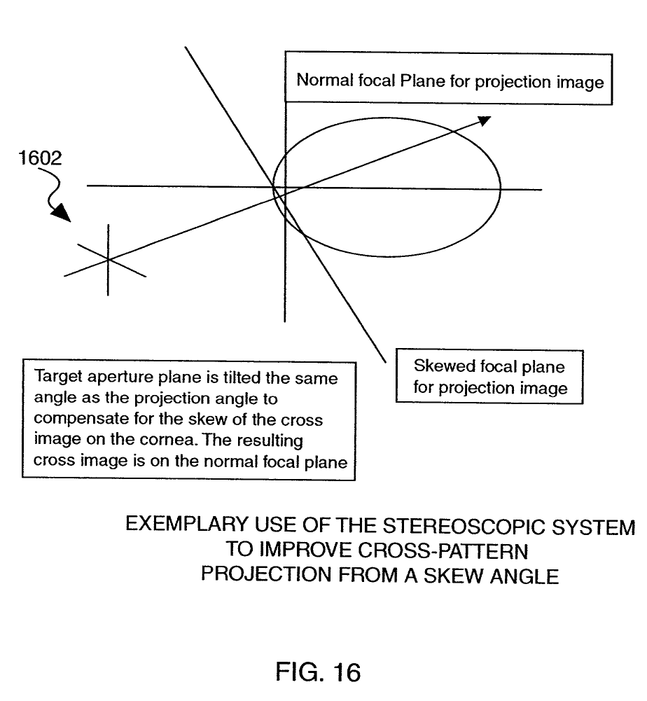

When shooting the projection pattern (e.g., cross pattern) from below, the depth of field of the images will tend to suffer due to the skew angle that the cross pattern lands on the cornea. FIG. 16 shows a compensation mechanism for the cross projection system to allow for a uniform depth of field when the pattern is projected on to the cornea. The mechanism is implemented by placing the target aperture plane 1602 30 degrees relative to the optical axis of the projection optics to produce an image on the cornea that is best focused in a normal plane

### Multi-Focal Capture of the Whole Eye

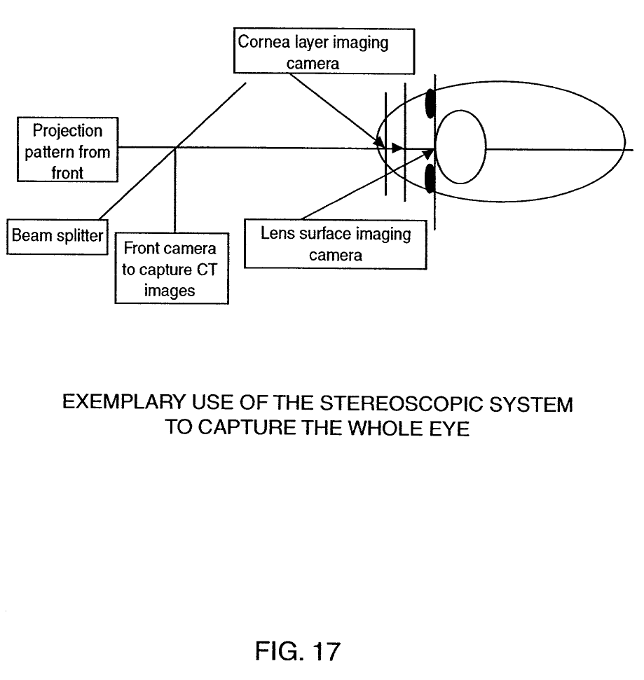

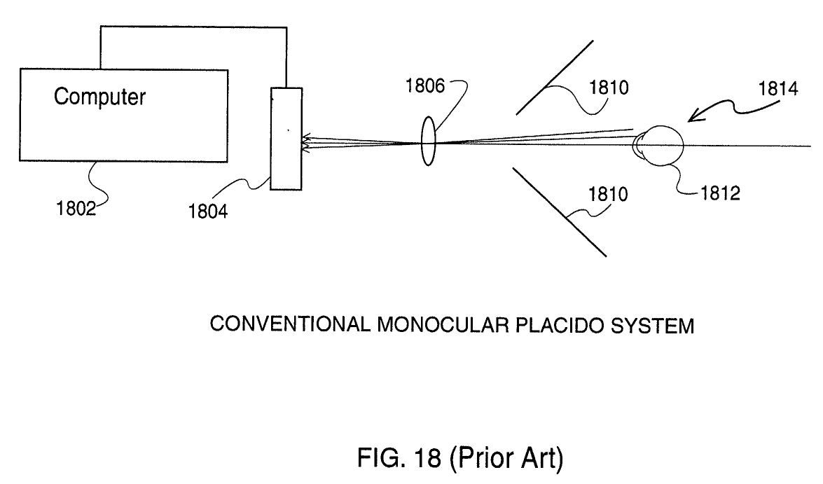

FIG. 17 shows a multi-focal capture of the entire eye. If the cross is shot from the front, the two side cameras and one front view camera may be used to capture the anterior corneal topography, cornea layer and lens surfaces. The projection pattern can be a cross, a cross plus dot array, dot array, or starburst pattern. The measurement can be done in a single shot with compromised accuracy, or selectively with high accuracy. Selective imaging means focusing the pattern at the corneal plane or the lens surface plane. For multiple segment acquiring using a quick sequence of acquisition, the cross pattern should be focused at about 3 mm after the apex, i.e., the entrance pupil plane.

While the invention has been described with reference to the exemplary embodiments thereof, those skilled in the art will be able to make various modifications to the described embodiments of the invention without departing from the true spirit and scope of the invention.

What is claimed is:

1. A stereoscopic eye measurement system, comprising:
a center camera centered as to said eye;
at least one camera at a skewed angle to the eye;
an infrared light source; and
a structured illumination pattern for a Placido;
wherein a pupil of said eye is enlarged, and said center camera and said at least one skewed angle camera capture an IR pupil image, a Placido image reflected off a cornea of said eye, and an image of an intersection of a corneal layer of said eye, an anterior segment of said eye, and a lens of said eye, with said structured illumination pattern projected onto said eye, to measure elements of said eye.

2. The stereoscopic eye measurement system according to claim 1, wherein:
said pupil is enlarged by the use of IR illumination in a dark environment during alignment and focusing.

3. The stereoscopic eye measurement system according to claim 1, wherein:
said system measures corneal thickness.

4. The stereoscopic eye measurement system according to claim 1, wherein:
said system measures corneal curvature.

5. The stereoscopic eye measurement system according to claim 1, wherein:
said system measures lens thickness.

6. The stereoscopic eye measurement system according to claim 1, wherein:
said system measures lens curvature.

7. The stereoscopic eye measurement system according to claim 1, wherein:
said system measures lens opacity.

8. The stereoscopic eye measurement system according to claim 1, wherein:
said system measures anterior chamber depth.

9. The stereoscopic eye measurement system according to claim 1, wherein:
said system measures an angle between a cornea of said eye and an iris of said eye.

10. The stereoscopic eye measurement system according to claim 1, wherein:
said system also measures corneal topography.

11. The stereoscopic eye measure measurement system according to claim 1, wherein:
a sequence of stereo images is obtained under ambient lighting varying from scotopic to photopic.

12. The stereoscopic eye measurement system according to claim 11, wherein:
said sequence of stereo images is used to measure the dynamic contouring and diameter measurement of an edge of said pupil of said eye.

13. The stereoscopic eye measurement system according to claim 11, wherein:
said sequence of stereo images is used to measure a locus of a center of said pupil of said eye varying in size.

14. The stereoscopic eye measurement system according to claim 11, wherein:
said sequence of stereo images is used to measure contouring of said edge of said pupil in three dimensions with a change in a diameter of said pupil in a z axis and along an optical axis direction.

15. The stereoscopic eye measurement system according to claim 11, wherein:
said sequence of stereo images is used to measure a geometry change of an iris of said eye with changes in a size of said pupil of said eye.

16. The stereoscopic eye measurement system according to claim 11, wherein:
said sequence of stereo images is used to measure a topography of a dynamic iris using a structured projection pattern image.

17. The stereoscopic eye measurement system according to claim 1, wherein:
said pupil of said eye is enlarged to at least 4.5 mm for measurement of said lens.

18. A method for stereoscopic measurement of an eye, comprising:
enlarging a pupil of said eye;
acquiring an IR pupil image, a structured illuminated Placido image reflected off a cornea of said eye, and an intersection image of a corneal layer of said eye, a lens of said eye, and an anterior segment of said eye with a structured projection pattern, using one camera centered on said eye and at least one camera at a skewed angle with respect to said eye; and
using said structured illumination pattern to measure elements of said eye.

19. The method for stereoscopic measurement of an eye according to claim 18, wherein said enlarging step comprises:
illuminating said pupil using infrared illumination in a dark environment.

20. The method for stereoscopic measurement of an eye according to claim 18, wherein said enlarging step comprises:
dilating said pupil.

21. The method for stereoscopic measurement of an eye according to claim 18, wherein:
said method measures corneal thickness.

22. The method for stereoscopic measurement of an eye according to claim 18, wherein:
said method measures corneal curvature.

23. The method for stereoscopic measurement of an eye according to claim 18, wherein:
said method measures lens thickness.

24. The method for stereoscopic measurement of an eye according to claim 18, wherein:
said method measures lens curvature.

25. The method for stereoscopic measurement of an eye according to claim 18, wherein:
said method measures lens opacity.

26. The method for stereoscopic measurement of an eye according to claim 18, wherein:
said method measures anterior chamber depth.

27. The method for stereoscopic measurement of an eye according to claim 18, wherein:
said method measures an angle between a cornea of said eye and an iris of said eye at their juncture.

28. The method for stereoscopic measurement of an eye according to claim 18, wherein:
said method measures corneal topography.

29. The method for stereoscopic measurement of an eye according to claim 18, wherein:
said method measures corneal thickness.

30. The method for stereoscopic measurement of an eye according to claim 18, wherein:
said pupil of said eye is enlarged to at least 4.5 mm for lens segment measurement.

31. The method for stereoscopic measurement of an eye according to claim 18, further comprising:
obtaining a sequence of stereo images under ambient lighting varying from scotopic to photopic.

32. The method for stereoscopic measurement of an eye according to claim 31, further comprising:
said sequence of stereo images is used to measure a dynamic contouring and diameter measurement of said pupil of said eye.

33. The method for stereoscopic measurement of an eye according to claim 31, further comprising:
said sequence of stereo images is used to measure a locus of a center of said pupil varying in size.

34. The method for stereoscopic measurement of an eye according to claim 31, further comprising:
said sequence of stereo images is used to measure a contouring of an edge of said pupil in three dimensions with a change in a diameter of said pupil in a z axis and along an optical axis direction.

35. The method for stereoscopic measurement of an eye according to claim 31, further comprising:
said sequence of stereo images is used to measure a geometry change of an iris of said eye with changes in a size of said pupil of said eye.

36. The method for stereoscopic measurement of an eye according to claim 18, further comprising:
said sequence of stereo images is used to measure a topography of a dynamic iris of said eye using a cross image sequence.

37. A method for stereoscopic measurement of an eye, comprising:
enlarging a pupil of said eye;
positioning a focal point of a camera centered on said eye and a focal point of at least one other camera skewed at a different angle to said eye so that said focal point of said centered camera and said focal point of said at least one skewed angle camera are within +/−3.0 mm of an entrance pupil plane;
acquiring a structured illumination pattern of a Placido reflected off a cornea of said eye, and a structured projection pattern intersection image projected through an opening on said Placido onto said eye, in said centered camera and said at least one skewed angle camera; and
using said structured illumination pattern to measure elements of said eye.

38. The method for stereoscopic measurement of an eye according to claim 37, wherein:
said pupil is enlarged to at least 4.5 mm.

39. The method for stereoscopic measurement of an eye according to claim 37, wherein:
said focal points of said centered camera and said at least one skewed angle camera are positioned at approximately 2.17 mm in front of an entrance pupil plane.

40. The method for stereoscopic measurement of an eye, according to claim 37, wherein:
said focal point of said centered camera and said focal point of said at least one skewed angle camera are positioned at approximately an entrance pupil plane of said eye.

41. The method for stereoscopic measurement of an eye, according to claim 37, wherein:
said focal point of said centered camera and said focal point of said at least one skewed angle camera are positioned approximately 1.5 mm in front of an entrance pupil plane of said eye.

42. The method for stereoscopic measurement of an eye, according to claim 37, wherein:
said focal point of said centered camera and said focal point of said at least one skewed angle camera are positioned approximately 3.5 mm behind an apex of said eye.

43. The method for stereoscopic measurement of an eye, according to claim 37, wherein:
said focal point of said centered camera and said focal point of said at least one skewed angle camera are determined by calculating a position of three stereo images and comparing said data with calibration data obtained by imaging a known planar target.

44. A method for stereoscopic measurement of an eye, comprising:
enlarging a pupil of said eye;
illuminating a structured Placido pattern to produce a structured illumination pattern;
reflecting said structured illumination pattern off said eye;
obtaining in a single acquisition said structured illumination pattern in one camera centered and at least one other camera skewed at a different angle, with respect to said eye; and
using said structured illumination pattern to measure at least one element of said eye.

45. The method for stereoscopic measurement of an eye according to claim 44, wherein:
said illuminating said structured Placido pattern uses an infrared illumination source.

46. The method for stereoscopic measurement of an eye according to claim 44, wherein said single acquisition is a single exposure of at least one camera.

47. The method for stereoscopic measurement of an eye according to claim 44, wherein said single acquisition uses multi-focal imaging for multiple segments of said eye.

48. The method for stereoscopic measurement of an eye according to claim 44, wherein said single acquisition is a few rapid exposures.

49. The method for stereoscopic measurement of an eye according to claim 44, wherein said measured at least one element of said eye is corneal topography.

50. The method for stereoscopic measurement of an eye according to claim 44, wherein said measured at least one element of said eye is a corneal layer.

51. The method for stereoscopic measurement of an eye according to claim 44, wherein said measured at least one element of said eye is an iris.

52. The method for stereoscopic measurement of an eye according to claim 44, wherein said measured at least one element of said eye is a lens.

53. The method for stereoscopic measurement of an eye according to claim 44, further comprising:
tilting a structured illumination target sufficient to compensate for a tilting of said projected light to achieve a normal projection focus on said eye.

54. A structured illumination pattern target for use in a stereoscopic eye measurement system, comprising:
a pattern on target material;
said pattern having an opening diameter from between 100 microns and 300 microns; and
said target material being thinner than said pattern opening diameter;
whereby illumination is projected through said pattern target to create a structured illumination pattern onto an eye.

55. The structured illumination pattern target according to claim 54, wherein:
said illumination is infrared illumination.

56. The structured illumination pattern target according to claim 54, wherein:
said target material is less than 300 microns in diameter.

57. The structured illumination pattern target according to claim 54, wherein:
said pattern is a cross pattern.

58. The structured illumination pattern target according to claim 54, wherein:
said pattern is a dot array.

59. The structured illumination pattern target according to claim 54, wherein:
said pattern is a dot+cross array.

60. The structured illumination pattern target according to claim 54, wherein:
said pattern is a starburst pattern.

61. A stereoscopic eye measurement system, comprising:
means for transmitting a structured illumination pattern through a structured illumination pattern target onto an eye;
means for enlarging a pupil of said eye;
means for obtaining an image of said structured illumination pattern reflected from said eye at an angle perpendicular to a normal to said eye;
means for obtaining at least one image of said structured illumination pattern reflected from said eye at a skewed angle to said eye; and
means for measuring at least one feature of said eye based on said reflected structured illumination pattern.

62. The stereoscopic eye measurement system according to claim 61, further comprising:
means for focusing said measurement system within approximately +/−3 mm of a pupil entrance plane of said eye.
